(19) United States
(12) Patent Application Publication
(10) Pub. No.: US 2019/0276409 A1
(43) Pub. Date: Sep. 12, 2019

(54) PROCESS FOR THE MANUFACTURE OF CARBOXYLIC ACIDS OR CARBOXYLIC ACID DERIVATIVES

(71) Applicant: SOLVAY SA, Brussels (BE)

(72) Inventor: Janis JAUNZEMS, Hannover (DE)

(21) Appl. No.: 16/347,960

(22) PCT Filed: Nov. 6, 2017
(86) PCT No.: PCT/EP2017/078259

§ 371 Date: May 7, 2019

(30) Foreign Application Priority Data
Nov. 7, 2016 (EP) 16197609.7

(51) Int. Cl. C07D 231/14

(52) U.S. Cl. CPC C07D 231/14

(57) ABSTRACT

This invention concerns a process for the manufacture of carboxylic acids or carboxylic acid derivatives and a process for the manufacture of agrochemically and pharmaceutically active compounds comprising the process for the manufacture of carboxylic acids or their derivatives. The process for the manufacture of carboxylic acids or carboxylic acid derivatives comprises the steps of: a) halogenating a compound of formula (I): R¹—C(O)—CHX₂, to obtain a compound of formula (II): R¹—C(O)—CX₂X′, b) transforming the compound of formula (II) in the presence of a compound A into a compound of formula (III): R′C(O)Z, wherein Z is a residue selected from the group consisting of —OH, —O⁻, —NR′R′. The process can optionally comprise additional steps.

This invention concerns a process for the manufacture of carboxylic acids or carboxylic acid derivatives and a process for the manufacture of agrochemically and pharmaceutically active compounds comprising the process for the manufacture of carboxylic acids or their derivatives.

Carboxylic acid and their derivatives, in particular 3-halomethylpyrazol-4-yl carboxylic acids and esters, are valuable intermediates in the synthesis of agrochemical and pharmaceutical active ingredients. Agrochemical active ingredients which contain 3-halomethylpyrazol-4-yl building blocks are, for example, 2′-[1,1′-bicycloprop-2-yl]-3-(difluoromethyl)-1-methylpyrazole-4-carboxanilide (Sedaxane), as described, for example, in WO2006015866, 3-(difluoromethyl)-1-methyl-N-[2-(3′,4′,5′-trifluorophenyl)phenyl]pyrazole-4-carboxamide (Fluxapyroxad), as described, for example, in WO2006087343, N-(3′,4′-Dichloro-5-fluorobiphenyl-2-yl)-3-(difluoromethyl)-1-methylpyrazole-4-carboxamide (Bixafen), as described, for example, in WO2003070705, 3-(Difluoromethyl)-1-methyl-N-[1,2,3,4-tetrahydro-9-(1-methylethyl)-1,4-methanonaphthalen-5-yl]-1H-pyrazole-4-carboxamide (Isopyrazam), as described, for example, in WO2004035589, (RS)—N-[9-(Dichloromethylen)-1,2,3,4-tetrahydro-1,4-methanonaphthalin-5-yl]-3-(difluoromethyl)-1-methyl-1H-pyrazole-4-carboxamide (Benzovindiflupyr), as described, for example, in WO07048556. Generally, 3-halomethylpyrazol-4-yl carboxylic acids, often obtained by hydrolysis of their esters, are converted into the carboxamides, for example after conversion into the 3-halomethylpyrazol-4-yl carboxylic acid halide. Other conversions, wherein the carboxamide is generated directly from the ester or acid, have also been described, such as in WO2012055864 and WO 2007/031323. All foregoing cited patent applications are hereby incorporated for all purposes.

Carboxylic acids can be obtained by oxidation of an activated methyl group, as described for example in CN105541716. When hypohalites are used for oxidation of the activated methyl group, a large amount, at least three equivalents, of hypohalite is necessary to convert the activated methyl group into a carboxylate salt. This results in a large volume of salt waste per mole carboxylate produced, which is often also difficult to treat in order to its organic impurities. As hypohalite solutions are often restricted in their upper concentration limit due to stability concerns, the waste volume is even higher per mole carboxylate produced.

It has been found that the process according to the present invention allows for the manufacture of a carboxylic acid or its derivative while avoiding a large amount of salt waste. The process shows good yields, lower waste and can be processed on a large scale.

The present invention thus concerns a process for the manufacture of a carboxylic acid or a carboxylic acid derivative of formula (III) R¹C(O)Z, which comprises the steps of

a) halogenating a compound of formula (I) R¹—C(O)—CHX₂, wherein X is selected form the group consisting of F, Cl, Br and I, and wherein each X in the compound of formula (I) is selected independently,

to obtain a compound of formula (II) R¹—C(O)—CX₂X′,

wherein X′ is selected form the group consisting of F, Cl, Br and I, and wherein X′ is the same as or different from each of X in the compound of formula (I),

wherein R¹ is a heterocyclic group which is optionally substituted

b) transforming the compound of formula (II) in the presence of a compound A into a compound of formula (III) R¹C(O)Z, wherein Z is a residue selected from the group consisting of —OH, —O⁻, —NR′R′ wherein R′ is independently selected from the group consisting of hydrogen or a C₁-C₁₂-alkyl group, C₂-C₆ alkenyl, aryl, cycloalkyl, aralkyl, heteroaryl, each of which is optionally substituted;

and wherein the process optionally further comprises a step c), wherein the compound of formula (III) is transformed into a compound of formula (IV) R¹COOH by treatment with an acid.

The invention further concerns a process comprising steps a), b) and optionally c), which further comprises a step of halogenating a compound of formula (V) R¹C(O)CH₃ with a halogenating agent to obtain a compound of formula (I).

The invention also concerns a process for the manufacture of an agrochemically or pharmaceutically active compound comprising steps a), b) and optionally c), which optionally further comprises a step of halogenating a compound of formula (V) R¹C(O)CH₃ with a halogenating agent to obtain a compound of formula (I).

In the present invention, designations in singular are in intended to include the plural; for example, “a solvent” is intended to denote also “more than one solvent” or “a plurality of solvents”.

In the context of the present invention, the term “comprising” is intended to include the meaning of “consisting of”.

In a first embodiment of the present invention, the invention concerns a process for the manufacture of a carboxylic acid or a carboxylic acid derivative of formula (III) R¹C(O)Z, which comprises the steps of

a) halogenating a compound of formula (I) R¹—C(O)—CHX₂, wherein X is selected form the group consisting of F, Cl, Br and I, and wherein each X in the compound of formula (I) is selected independently,

to obtain a compound of formula (II) R¹—C(O)—CX₂X′,

wherein X′ is selected form the group consisting of F, Cl, Br and I, and wherein X′ is the same as or different from each of X in the compound of formula (I),

wherein R¹ is a heterocyclic group which is optionally substituted

b) transforming the compound of formula (II) in the presence of a compound A into a compound of formula (III) R¹C(O)Z, wherein Z is a residue selected from the group consisting of —OH, —O⁻, —NR′R′ wherein R′ is independently selected from the group consisting of hydrogen or a C₁-C₁₂-alkyl group, C₂-C₆ alkenyl, aryl, cycloalkyl, aralkyl, heteroaryl, each of which is optionally substituted.

The definition “C₁-C₁₂-alkyl” group, or sub-ranges thereof, such as a C₁-C₄ or C₁-C₈ alkyl group, comprises the largest range defined herein for a C₁₋₁₂ alkyl group. Specifically, this definition comprises, for example, the meanings methyl, ethyl, n-propyl, isopropyl, n-, iso-, sec- and t-butyl, n-pentyl, n-hexyl, 1,3-dimethylbutyl, 3,3-dimethylbutyl, n-heptyl, n-nonyl, n-decyl, n-undecyl and n-dodecyl. Often, methyl, ethyl, n-propyl, isopropyl, n-, iso-, sec- and t-butyl are most preferred residues selected from the group C₁-C₁₂-alkyl. The term “cycloalkyl” generally intends to denote a C₃-C₁₀-cycloalkyl or C₃-C₈-cycloalkyl group, and generally denotes mono-, bi- or tricyclic hydrocarbon groups comprising 3 to 10 or 3 to 8 carbon atoms, especially 3 to 6 carbon atoms. Examples of monocyclic groups include cyclopropyl, cyclobutyl, cyclopentyl, cyclohexyl, cycloheptyl or cyclooctyl. Examples of bicyclic groups include bicyclo[2.2.1]heptyl, bicyclo[3.1.1]heptyl, bicyclo[2.2.2]octyl and bicyclo[3.2.1]octyl. Examples of tricyclic groups are adamantyl and homoadamantyl. According to the present invention, carbocyclic aromatic groups can have one or more rings in the aromatic system or attached thereto. Examples for a carbocyclic aromatic group are benzene, naphtalin, anthracen, phenantren, inden and pyren. The term “aromatic carbocycle” is also used for this group. According to the present invention, the term “heterocyclic group” can be aromatic or non-aromatic. Non aromatic heterocycles can have one or more rings in the system. Non-aromatic heterocycles are, for example, aziridine, azirine, oxirane, thiirane, azetidine, dihydroazete, diazetidine, oxetan, thietane, pyrrolidine, pyrroline, pyrazolidine, imidazolidine, pyrazoline, imidazoine, tetrahydrofurane, dioxolane, tetrahydrothiophene, oxathiolane, sulfolane, piperidine, piperazine, tetrahydropyran, pyran, dioxane, thiane, thiazine and pyrrolizine. Aromatic heterocycles can have one or more rings. Aromatic heterocycles are, for example, pyrrole, pyrazole, imidazole, triazole, tetrazole, furan, thiophene, oxazole, isoxazole, isothiazole, thiazole, oxadiazole, thiadiazole, pyridine, pyridazine, pyrimidine, pyrazine, triazine, indolizine, benzothiophene or benzofuran. R¹ is a heterocyclic group which is optionally substituted, and preferably is an aromatic heterocycle. More preferably, R¹ preferably is selected from the group consisting of pyrazole, pyrrole, furan, thiophene, oxazole, isoxazole, isothiazole, thiazole, oxadiazole, thiadiazole, pyridine, pyridazine, pyrimidine, pyrazine and triazine. Even more preferably, R¹ is a pyrazole or pyridine group. Pyrazole is the most preferred group R¹.

Each of the groups R¹ can optionally be substituted by one or more substituents of the group consisting of H, X″, COOR″, OR″, SR″, C(O)NR″₂, wherein R″ is selected from the group consisting of hydrogen, a C₁-C₁₂-alkyl group, CN, C₂-C₆ alkenyl, aryl, cycloalkyl, aralkyl, heteroaryl, each of which is optionally substituted, or a nitrogen protecting group, with the proviso that in C(O)NR″₂ both R″ may be the same or different, and X″ is selected from the group consisting of F, Br, Cl, and I.

In one preferred embodiment according to the present invention, the compound of formula (I) is the compound of formula (I_{q})

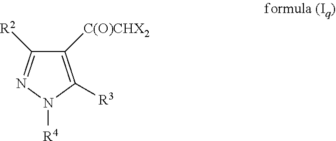

wherein R² is selected from the group consisting of C₁-C₄-alkyl groups which may be substituted by one, two or three halogen atoms selected from the group consisting of F, Cl and Br or by a CF₃ group. Preferably, R² is selected from the group consisting of CF₂Cl, CF₂H, CFCl₂, CFClH, CF₂Br, CF₂CF₃ and CF₃;

R³ is selected from the group consisting of H, X″, COOR″, OR″, SR″, C(O)NR″₂, wherein R″ selected from the group consisting of hydrogen, a C₁-C₁₂-alkyl group, CN, C₂-C₆ alkenyl, aryl, cycloalkyl, aralkyl, heteroaryl, each of which is optionally substituted, with the proviso that both R″ in C(O)NR′₂ may be the same or different, wherein X″ and R″ are defined as above. Preferably, R³ is H or X″, wherein H is preferred;

R⁴ is selected from the group consisting of H, C₁-C₁₂-alkyl, C₂-C₆ alkenyl, C₃-C₈ cycloalkyl, aryl, heteroaryl, aralkyl, each of which is optionally substituted; or R⁴ is a nitrogen protecting group. Preferably, R⁴ is a C₁-C₁₂-alkyl group, and it is most preferred that R⁴ is a methyl group. The term “nitrogen protecting group” intends to denote a group that is not cleaved by each of the reactions in the manufacturing method of the present invention, and is cleaved by other chemical methods (e.g., chemical methods such as hydrogenolysis, hydrolysis, electrolysis, photolysis as generally used in organic synthetic chemistry) into the N—H. Such protecting group can be selected from the commonly known or even well-known protecting groups known as amino-protecting groups. Examples include: alkyl carbamate based protecting groups such as tert-butyldiphenylsilyl, t-butyldimethylsilyl, methoxycarbonyl, ethoxycarbonyl, tert-butoxycarbonyl (Boc) groups; arylalkyl carbamate based protecting groups such as 9-fluorenylmethyloxycarbonyl (Fmoc); aryl sulfonamide based protecting groups such as benzenesulfonyl, p-toluenesulfonyl (Ts) group; amide based protecting groups such as carboxamido, acetamido, trifluoroacetamide (TFA), commonly known to persons skilled in the art according to synthetic chemistry reference books such as the “Protective Groups in Organic Synthesis” (T. W. Greene et. al, John Wiley & Sons, inc).

The halogenating agent for halogenating the compound of formula (I) in step a) preferably is selected from the group consisting of a a hypohalite, a base B and a halide, a halide, such as F₂, Cl₂, Br₂ and I, mixed (interhalogen) halides, such as BrCl, ClF₃, ClF, ICl, N-halosuccinimide, such as N-fluorosuccinimide, N-bromoosuccinimide, N-chlorosuccinimide and N-iodosuccinimide, thionyl halide, such as thionyl fluoride, thionyl bromide, thionyl chloride and thionyl iodide, phosphorous trihalide, such as PCl₃, PBr₃, PI₃, phosphorous pentahalide, such as PCl₅, PBr₅, Et₃N.3HF (TREAT-HF), (HF)ₓ.Pyr (Olahs reagent), Et₂NSF₃ (DAST), (Me₂N)₃S(Me)₃SiF₂ (TASF), PhIF₂, BF₃, XeF₂, CH₃COOF, CF₃COOF, CF₃OF, FOClO₃, N-Fluorobenzenesulfonimide chloride and sulfuryl chloride. The term “hypohalite” intends to denotes a hypohalous acid HOX or salts thereof, wherein the anion is selected from BrO⁻, FO⁻, IO⁻ and ClO⁻, and the cation is an alkali or earth alkali cation. Preferably, the hypohalite used in step a) is NaOBr or NaOCl. The combination “a base B and a halide” intends to denote a combination of F₂, Cl₂, Br₂ and I with an aqueous inorganic base B, such as alkali hydroxide or earth alkali hydroxide, or an organic base B, such as NEt₃. In the step a), a hypohalite or base B and halide are preferred halogenating agents, wherein aqueous solutions of hypochlorite, such as NaOCl, Ca(OBr)₂, NaOBr and Ca(ClO)₂ are most preferred.

In step b) of the process according to the present invention, the compound of formula (II) is transformed in the presence of a compound A into a compound of formula (III) R¹C(O)Z, wherein Z is a residue selected from the group consisting of —OR′, —O⁻, —NR′R′ wherein R′ is independently selected from the group consisting of hydrogen, C₁-C₁₂-alkyl, C₂-C₆ alkenyl, aryl, cycloalkyl, aralkyl, heteroaryl, each of which is optionally substituted by one or more substituents of the group consisting of H, X″, COOR″, OR″, SR″, C(O)NR″₂, wherein R″ is selected from the group consisting of hydrogen, a C₁-C₁₂-alkyl group, CN, C₂-C₆ alkenyl, aryl, cycloalkyl, aralkyl, heteroaryl, each of which is optionally substituted, with the proviso that in C(O)NR″₂ both R″ may be the same or different, and X″ is selected from the group consisting of F, Br, Cl, and I. Generally, compound A is selected from the group consisting of an alcohol with the formula R′OH, wherein R′ is as defined above, an aqueous solution of alkali or earth alkali salt, an alcoholate compound of formula R′O⁻M⁺ or (R′O⁻)₂M²⁺, wherein M is an alkali or earth alkali metal, and HNR′R′, wherein R′ may be the same or different, and wherein R′ is as defined above. In one aspect, compound A is an alcohol with the formula R′OH, wherein R′ is selected from the group consisting of C₁-C₁₂-alkyl, C₂-C₆ alkenyl, aryl, cycloalkyl, aralkyl and heteroaryl, and the compound (III) is a compound of formula (IIIa) R¹C(O)OR′ wherein R′ is selected from the group consisting of C₁-C₁₂-alkyl, C₂-C₆ alkenyl, aryl, cycloalkyl, aralkyl and heteroaryl. The presence of a base, such as K₂CO₃, in the reaction of (II) and compound A, when compound A is an alcohol with the formula R′OH, can be advantageous. In another aspect, the compound A is an aqueous solution of alkali or earth alkali salt, such as alkali or earth alkali carbonates, hydroxides or bicarbonates. Aqueous solutions of alkali or earth alkali hydroxide compounds, such as NaOH, Ca(OH)₂, LiOH, or KOH are preferred. When A is a solution of alkali or earth alkali salt, a carboxylate compound (IIIb) of formula R¹C(O)O⁻ is obtained, wherein the counter cation of (IIIb) is the cation contained in the alkali or earth alkali hydroxide compound. In one aspect, compound A is an alcoholate compound of formula R′O⁻M⁺ or (R′O⁻)₂M²⁺, wherein M is an alkali or earth alkali metal and R′ is defined as above. In the absence of water, Z in formula (III) R¹C(O)Z can be —OR′, wherein R′ is the residue in the alcoholate employed. In yet another aspect, compound A is a compound of formula HNR′R′, wherein R′ may be the same or different, and wherein R′ is as defined above. In one aspect, at least one of R′ is a hydrogen atom. In another preferred aspect, one R′ in compound HNR′R′ is hydrogen, and the other R′ is defined as a group Q, which is an optionally substituted aromatic carbocycle, non-aromatic or aromatic heterocyclic group, all of which can also be bi- or tricyclic, wherein one or more rings which are bound to the aromatic carbocycle or heterocyclic group can be non-aromatic. Generally, Q is selected from the group consisting of phenyl, naphtalene, 1,2,3,4-tetrahydronaphthalene, 2,3-dihydro-1H-indene, 1,3-dihydroisobenzofuran, 1,3-dihydrobenzo[c]thiophene, 6,7,8,9-tetrahydro-5H-benzo[7]annulene, thiophene, furan, thioazole, thiadiazole, oxazole, oxadiazole, pyridine, pyrimidine, triazine, tetrazine, thiazine, azepine and diazepine, each of which is optionally substituted. In one aspect, Q is a group of formula Q1

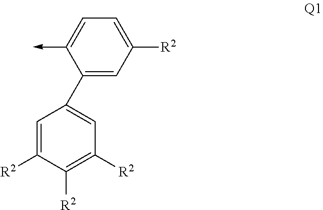

wherein each R² is independently selected from the group consisting of hydrogen or halogen, said halogen is especially chlorine or fluorine.

In another aspect, Q is a group of formula Q2

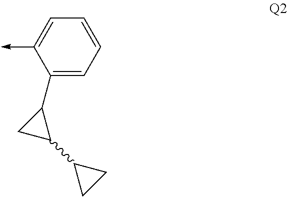

In another aspect, Q is a group of formula Q3

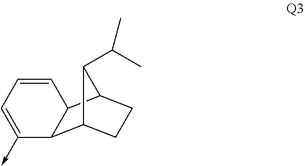

In yet another aspect, Q is a group of formula Q4

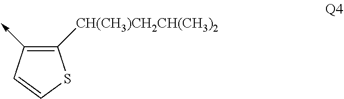

In one embodiment according to the present invention, the process according to claim 1, wherein the process further comprises a step c), wherein the compound of formula (III) is transformed into a compound of formula (IV) R¹COOH by treatment with an acid. Preferably, the compound (III) which is transformed in step c) into compound of formula (IV) R¹COOH by treatment with an acid is a compound of formula (Ma) or (IIIb), preferably (IIIb). In a most preferred aspect, compound A in step is an aqueous alkali or earth alkali hydroxide compound, such as NaOH, Ca(OH)₂, LiOH, or KOH, and a carboxylate compound (Mb) of formula R¹C(O)O⁻ is obtained, wherein the counter cation of (Mb) is the cation contained in the alkali or earth alkali hydroxide compound, and compound (Mb) is transformed into compound (IV) R¹COOH by treatment with an acid. The acid used in step c) preferably is selected from the group consisting of inorganic acids, such as H₂SO₄, HNO₃, HCl, HBr, HF, HI, H₃PO₄, H₃BO₃, HClO₄, and carboxylic acids, such as citric acid, acetic acid, propionic acid, malonic acid. HCl and H₂SO₄ are most preferred acids in step c).

In one preferred aspect according to the present invention, X and X′ are the same atom species in the compound of formula (II).

In one embodiment of the process for the manufacture of carboxylic acids or carboxylic acid derivatives, the process comprises a step d) of halogenating a compound of formula (V) R¹C(O)CH₃ with a halogenating agent to obtain a compound of formula (I).

In one embodiment according to the present invention, the halogenating agent for halogenating the compound of formula (V) in step d) is selected from the group consisting of a a halide, such as F₂, Cl₂, Br₂ and I, N-halosuccinimide, such as N-fluorosuccinimide, N-bromoosuccinimide, N-chlorosuccinimide and N-iodosuccinimide, thionyl halide, such as thionyl fluoride, thionyl bromide, thionyl chloride and thionyl iodide, phosphorous trihalide, such as PCl₃, PBr₃, PI₃, phosphorous pentahalide, such as PCl₅, PBr₅, Et₃N.3HF (TREAT-HF), (HF)ₓ.Pyr (Olahs reagent), Et₂NSF₃ (DAST), (Me₂N)₃S(Me)₃SiF₂ (TASF), PhIF₂, BF₃, XeF₂, CH₃COOF, CF₃COOF, CF₃OF, FOClO₃, N-Fluorobenzenesulfonimide chloride and sulfuryl chloride. The halogenating agent in step d) preferably is not a hypohalite, as this avoids the formation of high amounts of salt waste over a process comprising step d), a), b) and optionally c).

In one preferred embodiment, the process for the manufacture of a carboxylic acid or a carboxylic acid derivative of formula (III) R¹C(O)Z is process A which comprises the following steps in the order:

Firstly, step d) of halogenating a compound of formula (V) R¹C(O)CH₃ with a halogenating agent to obtain a compound of formula (I);

Secondly, step a) of halogenating a compound of formula (I) R¹—C(O)—CHX₂, wherein X is selected form the group consisting of F, Cl, Br and I, and wherein each X in the compound of formula (I) is selected independently,

to obtain a compound of formula (II) R¹—C(O)—CX₂X′,

wherein X′ is selected form the group consisting of F, Cl, Br and I, and wherein X′ is the same as or different from each of X in the compound of formula (I),

wherein R¹ is selected from the group consisting of an aliphatic, carbocyclic aromatic or heterocyclic group, each of which is optionally substituted;

and thirdly, step b) of transforming the compound of formula (II) in the presence of a compound A into a compound of formula (III) R¹C(O)Z, wherein Z is a residue selected from the group consisting of —OH, —O⁻, —NR′R′ wherein R′ is independently selected from the group consisting of hydrogen or a C₁-C₁₂-alkyl group, C₁-C₁₂-alkyl, C₂-C₆ alkenyl, aryl, cycloalkyl, aralkyl, heteroaryl, each of which is optionally substituted.

In the above preferred embodiment, R¹ preferably is a pyrazole group. The halogenating agent preferably is a chlorinating agent. Compound A preferably is an aqueous solution of alkali or earth alkali hydroxide compounds, such as NaOH, Ca(OH)₂, LiOH, or KOH.

In another preferred embodiment, the process A of firstly step d), secondly step a) and thirdly step b) comprises a fourth step c), wherein the compound of formula (III) is transformed into a compound of formula (IV) R¹COOH by treatment with an acid.

In one preferred aspect of process A, the halogenating agent of step a) is selected from the group consisting of a halide, such as F₂, Cl₂, Br₂ and I, N-halosuccinimide, such as N-fluorosuccinimide, N-bromoosuccinimide, N-chlorosuccinimide and N-iodosuccinimide, thionyl halide, such as thionyl fluoride, thionyl bromide, thionyl chloride and thionyl iodide, phosphorous trihalide, such as PCl₃, PBr₃, PI₃, phosphorous pentahalide, such as PCl₅, PBr₅, Et₃N.3HF (TREAT-HF), (HF)ₓ.Pyr (Olahs reagent), Et₂NSF₃ (DAST), (Me₂N)₃S(Me)₃SiF₂ (TASF), PhIF₂, BF₃, XeF₂, CH₃COOF, CF₃COOF, CF₃OF, FOClO₃, N-Fluorobenzenesulfonimide chloride and sulfuryl chloride. The halogenating agent in step d) of process A preferably is not a hypohalite.

When R¹ is the fragment of formula R_{q}, the manufacture of a compound of R_{q}C(O)CH₃ is described in CN105541716.

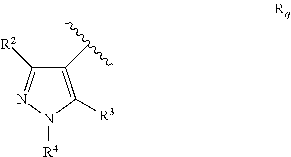

In one embodiment according to the present invention, both X in compound (I) obtained by step d) are Cl or both X in compound (I) obtained by step d) are Br.

In one preferred embodiment, the process for the manufacture of a carboxylic acid or a carboxylic acid derivative of formula (III) R¹C(O)Z is process A which comprises the following steps in the order:

Firstly, step d) of halogenating a compound of formula (V_{q}) R¹C(O)CH₃, wherein R¹ is R_{q}, with a halogenating agent to obtain a compound of formula (I_{q})

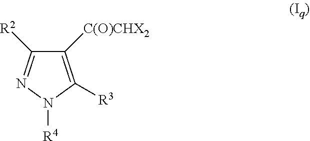

Secondly, step a) of halogenating a compound of formula (I_{q}),

to obtain a compound of formula (II_{q}),

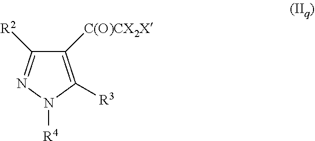

wherein the preferred halogenating agent in step a) is a hypohalite, preferably NaOCl or NaOBr;

and thirdly, step b) of transforming the compound of formula (II_{q}) in the presence of a compound A into a compound of formula (III_{q}), wherein compound A is selected from the group consisting of aqueous alkali or earth alkali hydroxide compounds, such as NaOH, Ca(OH)₂, LiOH, or KOH, wherein NaOH is preferred

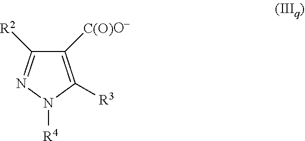

In another preferred embodiment, the above process A of firstly step d) on formula (V_{q}), secondly step a) on formula (I_{q}) and thirdly step b) on compound (II_{q}) to obtain compound (III_{q}) comprises a fourth step c), wherein the compound of formula (III_{q}) is transformed into a compound of formula (IV_{q})

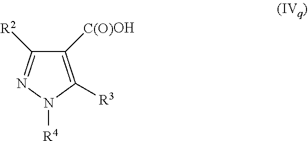

by treatment with an acid. The acid preferably is selected from the group consisting of HCl, HBr, HNO₃ and H₂SO₄, wherein HCl is most preferred.

In an even more preferred aspect of the process A, comprising successively step d), step a), step b) and step c), compound A is present in step a) such that step a) and step b) are performed in direct succession, for example when an aq. NaOH/NaOCl solution is used in step a).

The invention further concerns a process for the manufacture of an agrochemically or pharmaceutically active compound which comprises the process for the manufacture of a carboxylic acid or a carboxylic acid derivative, which comprises steps a) and b), optionally step c) and further optionally step d). An agrochemically or pharmaceutically active compound can, for example, be obtained by converting a compound of formula (IV) obtained by the process according to the present invention into a carboxylic acid halide or anhydride, and reacting the carboxylic acid halide or anhydride with a primary or secondary amine to obtain a carboxamide which is an agrochemically or pharmaceutically active compound. Such reactions are known, for example, from WO2003070705. In such a process for the manufacture of an agrochemical compound, for example compounds such as N-(3′,4′-Dichlor-5-fluorbiphenyl-2-yl)-3-(difluormethyl)-1-methylpyrazol-4-carboxamid, 3-(difluoromethyl)-1-methyl-N-[2-(3′,4′,5′-trifluorophenyl)phenyl]pyrazole-4-carboxamide, N-(2-Bicyclopropyl-2-ylphenyl)-3-difluoromethyl-1-methyl-1H-pyrazol-4-carboxylic acid amide, 3-(Difluormethyl)-1-methyl-N-[1,2,3,4-tetrahydro-9-(1-methylethyl)-1,4-methanonaphthalen-5-yl]-1H-pyrazol-4-carboxamid or N-[(1RS,4SR)-9-(dichloromethylidene)-1,2,3,4-tetrahydro-1,4-methanonaphthalen-5-yl]-3-(difluoromethyl)-1-methyl-1H-pyrazole-4-carboxamide (and isomers) are obtained.

The new process according to the present invention allow for efficient syntheses of carboxylic acids or a carboxylic acid derivatives, which are useful intermediates for, e.g., agrochemical and pharmaceutical compounds. Departing from easily accessible starting materials, such as methyl ketones, the carboxylic acids or carboxylic acid derivatives can be obtained while avoiding a high amount of salt waste which often also is difficult to dispose of and/or recycle due to organic impurities.

The invention further concerns a compound of formula (I) R¹—C(O)—CHX₂, wherein X is selected form the group consisting of F, Cl, Br and I, and wherein both X are the same as or different from each other, and wherein R¹ is a heterocyclic group which is optionally substituted. R¹ is preferably selected from the group consisting of pyrazole, pyrrole, furan, thiophene, oxazole, isoxazole, isothiazole, thiazole, oxadiazole, thiadiazole, pyridine, pyridazine, pyrimidine, pyrazine and triazine. Even more preferably, R¹ is a pyrazole or pyridine group. Pyrazole is the most preferred group R¹. R¹ can optionally be substituted by one or more substituents of the group consisting of H, X″, COOR″, OR″, SR″, C(O)NR″₂, wherein R″ is selected from the group consisting of hydrogen, a C₁-C₁₂-alkyl group, CN, C₂-C₆ alkenyl, aryl, cycloalkyl, aralkyl, heteroaryl, each of which is optionally substituted, or a nitrogen protecting group, with the proviso that in C(O)NR″₂ both R″ may be the same or different, and X″ is selected from the group consisting of F, Br, Cl, and I. In a preferred aspect, both X in (I) are Cl. In another preferred aspect, both X in (I) are Br. The most preferred compound of formula (I) is the compound of formula (I_{q}) as described above. The compounds of formula (I) are useful as intermediates in the manufacture of compound of formula (II) and/or (III), which are pharmaceutical or agrochemical active ingredients or intermediates in manufacturing processes for pharmaceutical or agrochemical active ingredients.

Should the disclosure of any patents, patent applications, and publications which are incorporated herein by reference conflict with the description of the present application to the extent that it may render a term unclear, the present description shall take precedence.

The following examples are intended to further explain the invention without limiting it.

### EXAMPLE 1

### Chlorination of 1-(3-(difluoromethyl)-1-methyl-1H-pyrazol-4-yl)ethanone to Obtain 2,2-dichloro-1-(3-(difluoromethyl)-1-methyl-1H-pyrazol-4-yl)ethanone

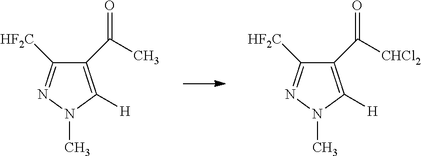

3 eq SO₂Cl₂ are mixed with dichloromethane (DCM) and cooled to 0° C. 1 eq 1-(3-(difluoromethyl)-1-methyl-1H-pyrazol-4-yl)ethanone in DCM is added and the mixture is slowly warmed over 2 hours to 20° C. The reaction is then warmed to 50° C. for 4 h, quenched with ice water and ethyl acetate is added. The organic phase is separated, washed with water and brine and dried over Na₂SO₄. 2,2-dichloro-1-(3-(difluoromethyl)-1-methyl-1H-pyrazol-4-yl)ethanone is obtained after removal of the volatiles.

### EXAMPLE 2

### 3-(difluoromethyl)-1-methyl-1H-pyrazole-4-carboxylic Acid

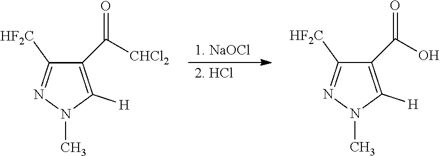

2,2-dichloro-1-(3-(difluoromethyl)-1-methyl-1H-pyrazol-4-yl)ethanone obtained from example 1 is mixed at 22° C. with 2 eq NaOH (10% in water) and 1.1. eq of a 8% sodium hypochlorite aqueous solution, and then stirred at 70° C. for 2 hours. The reaction solution is quenched with ice water, then saturated sodium sulfite aqueous solution is added. After addition of 3.3 eq 10% HCl, the aqueous phase is extracted twice with isopropyl acetate. The solvent is removed, and 3-difluoromethyl)-1-methyl-1H-pyrazole-4-carboxylic acid is obtained.

1. A process for the manufacture of a carboxylic acid or a carboxylic acid derivative of formula (III) R¹C(O)Z, the process comprising the steps of
a) halogenating a compound of formula (I) R¹—C(O)—CHX₂, wherein X is selected from the group consisting of F, Cl, Br and I, and wherein each X in the compound of formula (I) is selected independently,
to obtain a compound of formula (II) R¹—C(O)—CX₂X′,
wherein X′ is selected from the group consisting of F, Cl, Br and I, and wherein X′ is the same as or different from each of X in the compound of formula (I),
wherein R¹ is a heterocyclic group, which is optionally substituted;
b) transforming the compound of formula (II) in the presence of a compound A into the compound of formula (III) R¹C(O)Z, wherein Z is a residue selected from the group consisting of —OH, —O⁻, —NR′R′ wherein each R′ is independently selected from the group consisting of hydrogen, C₁-C₁₂-alkyl, C₂-C₆ alkenyl, aryl, cycloalkyl, aralkyl, and heteroaryl, each of which is optionally substituted, and wherein compound A is selected from the group consisting of an alcohol with the formula R′OH, wherein R′ is as defined above, an aqueous solution of alkali or earth alkali salt, an alcoholate compound of formula R′O⁻M⁺ or (R′O⁻)₂M²⁺, wherein M is an alkali or earth alkali metal, and HNR′R′, wherein R′ may be the same or different, and wherein R′ is as defined above.

2. The process according to claim 1, wherein the process further comprises a step c), wherein the compound of formula (III) is transformed into a compound of formula (IV) R¹COOH by treatment with an acid.

3. The process according to claim 1, wherein the compound of formula (I) is halogenated with a halogenating agent selected from the group consisting of a hypohalite; a base B, wherein Base B is an organic or inorganic base, a halide, thionyl chloride, and sulfuryl chloride.

4. The process according to claim 3, wherein the hypohalite is selected from the group consisting of NaOCl, Ca(ClO)₂, Ca(BrO)₂ and NaOBr.

5. The process according to claim 3, wherein the base B is selected from an aqueous alkali or earth alkali hydroxide and the halide is selected from bromine or chlorine.

6. The process according to claim 1, wherein R¹ is selected from the group consisting of pyrazole, pyrrole, furan, thiophene, oxazole, isoxazole, isothiazole, thiazole, oxadiazole, thiadiazole, pyridine, pyridazine, pyrimidine, pyrazine and triazine, each of which is optionally substituted.

7. The process according to claim 6, wherein the compound of formula (I) is the compound of formula (I_{q}) 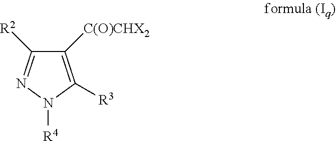
wherein R² is selected from the group consisting of C₁-C₄-alkyl groups which may be substituted by one, two or three halogen atoms selected from the group consisting of F, Cl and Br or by a CF₃ group,
R³ is selected from the group consisting of H, X″, COOR″, OR″, SR″, C(O)NR″₂, wherein R″ selected from the group consisting of hydrogen, a C₁-C₁₂-alkyl group, CN, C₂-C₆ alkenyl, aryl, cycloalkyl, aralkyl, and heteroaryl, each of which is optionally substituted, with the proviso that both R″ in C(O)NR″₂ may be the same or different, wherein X″ and R″ are defined as above,
R⁴ is selected from the group consisting of H, C₁-C₁₂-alkyl, C₂-C₆ alkenyl, C₃-C₈ cycloalkyl, aryl, heteroaryl, and aralkyl, each of which is optionally substituted; or R⁴ is a nitrogen protecting group.

8. The process according to claim 7, wherein the compound of formula (I) is a compound of formula (I_{q}), wherein R² is selected from the group consisting of CF₂Cl, CF₂H, CFCl₂, CFClH, CF₂Br, CF₂CF₃ and CF₃.

9. The process according to claim 7, wherein R³ is selected from the group consisting of H, X″, C₁-C₁₂-alkyl group, and CN, and R⁴ is a C₁-C₁₂-alkyl group.

10. The process according to claim 1, which further comprises a step d) of halogenating a compound of formula (V) R¹C(O)CH₃ with a halogenating agent to obtain a compound of formula (I).

11. The process according to claim 10, wherein formula (V) is a compound of formula (V_{q}) R¹C(O)CH₃, wherein R¹ is R_{q}

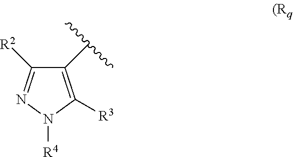

12. The process according to claim 10, wherein both X in compound (I) obtained by step d) are Cl or both X in compound (I) obtained by step d) are Br.

13. The process according to claim 10, wherein the halogenating agent used to obtain the compound of formula (I) is selected from the group consisting of a halide, thionyl chloride and sulfuryl chloride.

14. A process for the manufacture of an agrochemically or pharmaceutically active compound which comprises the process according to claim 1.

15. A compound of formula (I) R¹—C(O)—CHX₂, wherein X is selected from the group consisting of F, Cl, Br and I, and wherein each X are the same or different from each other, and wherein R¹ a heterocyclic group which is optionally substituted.

16. The process according to claim 9, wherein R⁴ is a methyl group.
